(19) United States
(12) Patent Application Publication
(10) Pub. No.: US 2019/0374269 A1
(43) Pub. Date: Dec. 12, 2019

(54) ATTACHMENT DEVICE AND METHOD FOR USE

(71) Applicant: Stout Medical Group, LP., Quakertown, PA (US)

(72) Inventors: Robert A. KIEFER, Quakertown, PA (US); E. Skott GREENHALGH, Gladwyne, PA (US)

(73) Assignee: Stout Medical Group, L.P., Quakertown, PA (US)

(21) Appl. No.: 16/510,608

(22) Filed: Jul. 12, 2019

Related U.S. Application Data
(63) Continuation-in-part of application No. 16/033,029, filed on Jul. 11, 2018.
(60) Provisional application No. 62/595,217, filed on Dec. 6, 2017.

(51) Int. Cl. A61B 17/86; A61B 17/70

(52) U.S. Cl. CPC A61B 17/863; A61B 2017/8655; A61B 17/70; A61B 17/8685

(57) ABSTRACT

Attachment devices and methods of using the same are disclosed. The attachment devices can be longitudinally expandable and/or compressible. The attachment devices can have an increasable and/or decreasable length.

### CROSS-REFERENCE TO RELATED APPLICATIONS

This application is a continuation-in-part of U.S. patent application Ser. No. 16/033,029, filed Jul. 11, 2018, which claims priority to U.S. Provisional Application No. 62/595,217, filed Dec. 6, 2017, which are incorporated by reference herein in their entireties.

### BACKGROUND

### 1. Technical Field

Attachment devices and methods of using the same are disclosed. More specifically, longitudinally expandable and/or contractible attachment devices for use in bone and methods of using the same are disclosed.

### 2. Background of the Art

Broken bones, such as compression fractures of one or more vertebrae in the spine, may be treated with internal fixation. Any indication needed spinal stability can also be treated by internal fixation. Examples include scoliosis, kyphosis, spondylothisthesis and rotation, segmental instability, such as disc degeneration and fracture caused by disease and trauma and congenital defects, and degeneration caused by tumors.

Internal fixation in the spine is often accomplished by first screwing fixation screws into the pedicles and vertebral bodies of the vertebrae. The fixation screws are then typically attached to a rigid fixation rod or plate that provide support between one or more weakened vertebra. This support often immobilizes the vertebra to which the fixation screws have been inserted.

Current screws have fixed dimensions. When screws are used in orthopedic applications, the screw length in softer bone (e.g., cancellous bone) is typically maximized. The length and shape of the bone where the screw is to be placed currently defines the screw diameter and length that should be used. In spinal applications, screw length is governed by vertebral body size. Doctors select the screw diameter based on the pedicle dimensions and the screw length on the length of the pedicle and the vertebral body. Typically, longer and larger diameter screws are used in the lower lumbar region.

Screw length and screw diameter directly govern screw “purchase” and long term durability in the bone. Longer and larger diameter screws anchor better under tensile loads. Longer and larger diameter screws also better resist movement inside the bone when subjected to long term dynamic motion.

Current screws have a fixed length. Accordingly, a need currently exists to have an axially adjustable attachment device with an increasable and/or decreasable length to aid with the insertion and/or removal of the attachment device, and to increase the amount of bone that current attachment devices can attach to (or “purchase”).

### BRIEF SUMMARY

This disclosure relates generally to attachment devices that can be expanded and/or contracted.

More specifically, longitudinally expandable and/or contractible attachment devices for use in bone and methods of using the same are disclosed.

Attachment devices are disclosed. For example, an attachment device is disclosed having a device longitudinal axis. The attachment device can have a longitudinally expandable length comprising a non-deformable frame having a first shaft and a second shaft. The first shaft can be telescopable within the second shaft.

Methods for deploying attachment devices are disclosed. For example, a method is disclosed for deploying an attachment device having a device longitudinal axis. The method can include inserting the attachment device into bone. The method can include longitudinally expanding a longitudinally expandable length of the attachment device.

### BRIEF DESCRIPTION OF THE DRAWINGS

The drawings shown and described are exemplary embodiments and non-limiting. Like reference numerals indicate identical or functionally equivalent features throughout.

FIGS. 1A-1C illustrate a variation of an attachment device and a method for longitudinally expanding and contracting the device. FIG. 1A illustrates a side view of the attachment device in a longitudinally unexpanded configuration. FIG. 1B illustrates a side view of the attachment device in a longitudinally expanded configuration. FIG. 1C illustrates a variation of a longitudinal cross-sectional view of the attachment device of FIG. 1A taken along line 1C-1C.

FIGS. 2A-2C illustrate a variation of an attachment device and a method for longitudinally expanding and contracting the device. FIG. 2A illustrates a side view of the attachment device in a longitudinally expanded configuration. FIG. 2A illustrates a variation of a longitudinal cross-sectional view of the attachment device of FIG. 2A in a longitudinally unexpanded configuration. FIG. 2C illustrates a variation of a longitudinal cross-sectional view of the attachment device of FIG. 2A taken along line 2C-2C.

FIGS. 3A-3C illustrate a variation of an attachment device and a method for longitudinally expanding and contracting the device. FIG. 3A illustrates a side view of the attachment device in a longitudinally expanded configuration. FIG. 3B illustrates a variation of a longitudinal cross-sectional view of the attachment device of FIG. 3A taken along line 3B-3B. FIG. 3B illustrates a variation of a transverse cross-sectional view of the attachment device of FIG. 3B taken along line 3C-3C.

FIGS. 4A and 4B illustrate a variation of an attachment device and a method for longitudinally expanding and contracting the device. FIG. 4A illustrates a side view of the attachment device in a longitudinally expanded configuration. FIG. 4B illustrates a perspective view of the attachment device in a longitudinally expanded configuration.

FIGS. 5A-5F illustrate a variation of an attachment device and a method for longitudinally expanding and contracting the device. FIG. 5A illustrates a side view of the attachment device in a longitudinally unexpanded configuration. FIG. 5B illustrates a side view of the attachment device in a longitudinally expanded configuration. FIG. 5C illustrates a variation of a longitudinal cross-sectional view of the attachment device of FIG. 5A taken along line 5C-5C. FIG. 5D is a magnified view of the attachment device of FIG. 5C at section 5D-5D. FIG. 5E illustrates a magnified view of a stop of a locking mechanism. FIG. 5F illustrates a variation of a transverse cross-sectional view of the attachment device of FIG. 5C taken along line 5F-5F.

FIGS. 6A-6D illustrate a variation of an attachment device and a method for longitudinally expanding and contracting the device. FIG. 6A illustrates a side view of the attachment device in a longitudinally unexpanded configuration. FIG. 6B illustrates a side view of the attachment device in a longitudinally expanded configuration. FIG. 6C illustrates a perspective view of the attachment device in a longitudinally expanded configuration. FIG. 6D illustrates a variation of a transverse end view of the attachment device of FIG. 6A taken along line 6B-6B.

FIGS. 7A-7D illustrate a variation of an attachment device having a variation of a locking mechanism and a method for longitudinally expanding and contracting the device. FIG. 7A illustrates a perspective view of the attachment device in a longitudinally unexpanded configuration removably attached to a variation of an attachment tool. FIG. 7B illustrates a side view of the attachment device in a longitudinally expanded configuration, FIG. 7C illustrates a magnified view of a stop of a locking mechanism. FIG. 7D illustrates a side view of a variation of the attachment device of FIG. 7B with the distal shaft transparent so that the locking mechanism can be visible in an engaged configuration when the device is in a longitudinally expanded configuration.

FIGS. 8A-8C illustrate a variation of an attachment device having a variation of a locking mechanism and a method for longitudinally expanding and contracting the device. FIG. 8A illustrates a perspective view of a variation of a proximal shaft of the attachment device having the locking mechanism. FIG. 8B illustrates a close-up perspective view of the locking mechanism of FIG. 8A at section 8B-8B. FIG. 8C illustrates a side view of a variation of the attachment device of FIGS. 8A and 8B with the distal shaft transparent so that the locking mechanism can be visible in an engaged configuration when the device is in a longitudinally expanded configuration.

FIGS. 9A and 9B illustrate a variation of an attachment device having a variation of a locking mechanism and a method for longitudinally expanding and contracting the device. FIG. 9A illustrates an exploded perspective view of the attachment device with the locking mechanism, FIG. 9B illustrates a side view of a variation of the attachment device of FIG. 9A with the distal shaft transparent so that the locking mechanism can be visible in an engaged configuration when the device is in a longitudinally expanded configuration.

FIGS. 10A-10E illustrate a variation of an attachment device having a variation of a locking mechanism and a method for longitudinally expanding and contracting the device. FIG. 10A illustrates a perspective view of a variation of a proximal shaft of the attachment device having the locking mechanism. FIG. 10B illustrates a magnified view of the proximal shaft of FIG. 10A at section 10B-10B. FIG. 10C illustrates a side view of the proximal shaft of FIG. 10A. FIG. 10D illustrates a top view of the proximal shaft of FIG. 10A. FIG. 10E illustrates a side view of a variation of an attachment device having the proximal shaft of FIG. 10A and with the distal shaft transparent so that the locking mechanism can be visible in an engaged configuration when the device is in a longitudinally expanded configuration.

FIGS. 11A-11E illustrate a variation of an attachment device having a variation of a locking mechanism and a method for longitudinally expanding and contracting the device. FIG. 11A illustrates a side view of the locking mechanism. FIG. 11B illustrates a side view of the locking mechanism. FIG. 11C illustrates a side view of a variation of the attachment device having the locking mechanism of FIGS. 11A and 11B with the distal shaft transparent so that the locking mechanism can be visible in an engaged configuration when the device is in a longitudinally expanded configuration. FIG. 11D illustrates a magnified view of the attachment device of FIG. 11C at section 11D-11D. FIG. 11E illustrates a side view of the attachment device of FIG. 11C with the distal shaft not transparent.

FIGS. 12A and 12B illustrate schematic side views of the attachment devices of FIGS. 1A-11E implanted in various vertebrae in expanded configurations. FIG. 12A illustrates a variation of an attachment device implanted in a vertebra when in an expanded configuration. FIG. 12B illustrates a variation of two attachment devices implanted in two different vertebrae when each attachment device is in an expanded configuration.

### DETAILED DESCRIPTION

Lengthening attachment devices (also referred to as erectile attachment devices, erectile devices, erecting devices, erectable devices, telescoping attachment devices, telescoping devices, telescopable devices and other similar terms) for use in bone are disclosed. The devices can be expandable and/or contractible along a device longitudinal axis. The devices can increase in length and/or decrease in length along the device longitudinal axis with or without expansion and/or contraction along a transverse axis perpendicular to the device longitudinal axis. The devices can increase in length and/or decrease in length along the device longitudinal axis concurrently with or independent from expansion and/or contraction along a transvers axis perpendicular to the device longitudinal axis. The devices can be inserted into bone at a first length and then expanded from the first length to a second length. During expansion, the device tip can move away from the base of the device. The base of the device can remain in a fixed position during expansion of the device. The devices can have one or more expanded configurations (also referred to as erect configurations, erected configurations, lengthened configurations and other similar terms) and one or more unexpanded configurations (e.g., also referred to as non-erect configurations, non-erected configurations, non-expanded configurations, sleeved configurations, contracted configurations and other similar terms). The devices can have a locking mechanism to “lock” the device in an expanded configuration. The devices can be a screw, for example, a pedicle screw. The screws are also referred to as erectile screws, erecting screws, erectable screws, telescoping screws, telescopable screws, expandable screws, contractible screws and other similar terms.

The attachment devices can have a distal shaft moveable relative to a proximal shaft. The proximal and distal shafts can be moved (e.g., rotated and/or translated) in unison during initial insertion of the device into bone. The distal shaft can be moved (e.g., rotated and/or translated) independently of the proximal shaft during expansion and/or contraction of the device.

The attachment devices can have a first shaft and a second shaft moveable relative to the first shaft. The first and second shafts can be moved (e.g., rotated and/or translated) in unison during initial insertion of the device into bone. The second shaft can be moved (e.g., rotated and/or translated) independently of the first shaft during expansion and/or contraction of the device.

The attachment devices can have a locking mechanism that can lock the device into an unexpanded configuration, a partially expanded configuration, a fully expanded configuration, or any combination thereof. The locking mechanism can prevent the distal/second shaft from being expanded completely out of the proximal/first shaft. The locking mechanism can be a ratcheting system, a mechanism that produces a friction fit between the two shafts in one or multiple positions, or combinations thereof.

### System and Apparatus

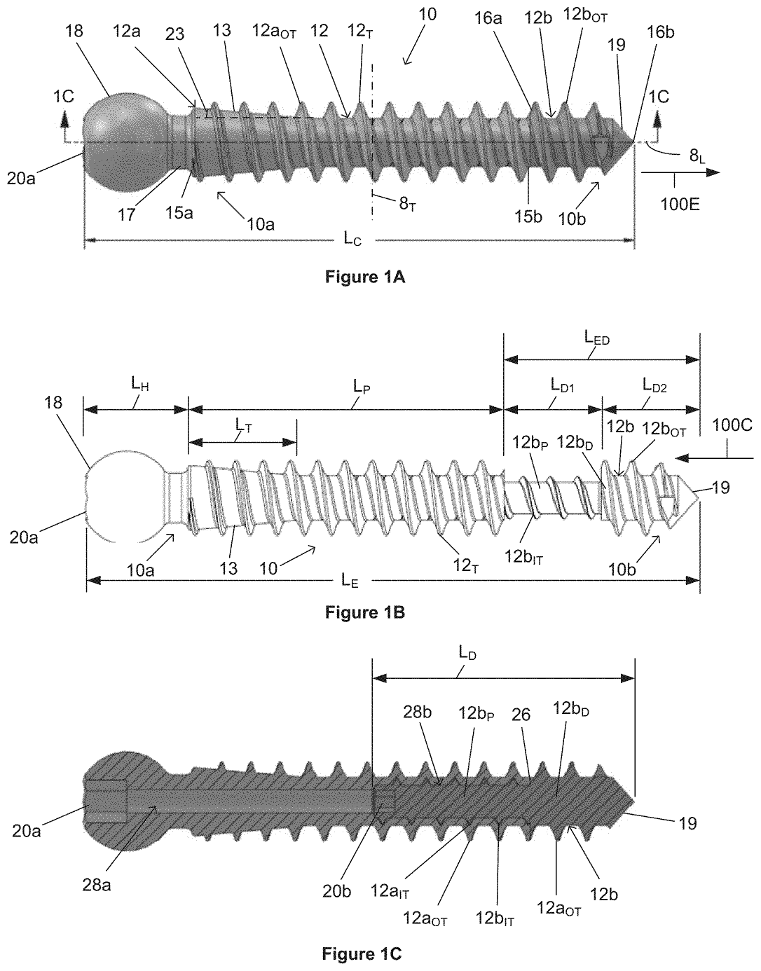

FIG. 1A illustrates a variation of an attachment device 10 that can be longitudinally adjusted (e.g., expanded and/or contracted) before, during, and/or after insertion into a medium. The medium can be tissue (e.g., bone). The device 10 can be longitudinally expandable and/or contractible along a device longitudinal axis 8_{L}. The device longitudinal axis 8_{L}, can be a center longitudinal axis of the device 10. The device longitudinal axis 8_{L} can be straight or curved. The device longitudinal axis 8_{L} can be perpendicular to a device transverse axis 8_{T}. The device transverse axis 8_{T} can be a center transverse axis of the device 10. The device transverse axis 8_{T} can be straight or curved.

The device 10 can be longitudinally expandable and/or contractible along the device longitudinal axis 8_{L} with or without being simultaneously or sequentially expandable and/or contractible along the device transverse axis 8_{T}. The device 10 can be lengthened (e.g., longitudinally expanded) and/or shortened (e.g., longitudinally contracted). The device 10 can have an increasable length when the device 10 is in a fully or partially contracted configuration, where “fully contracted” is also referred to throughout as “unexpanded.” The term “contracted” throughout can be the initial configuration of the device 10 (e.g., a first configuration), and/or the term “contracted” throughout can imply that the device 10 was previously in an expanded configuration. The device 10 can have a decreasable length when the device 10 is in a fully or partially expanded configuration. When the device 10 is lengthened, one or more sections of the device 10 can have a transverse dimension (e.g., diameter, width, height) that decreases. When the device 10 is shortened, one or more sections of the device 10 can have a transverse dimension (e.g., diameter, width, height) that increases. The device 10 can increase and/or decrease in length only such that the device 10 is not transversely adjustable (e.g., is not expandable or contractible along the device transverse axis 8_{T}).

The device 10 can be longitudinally lengthened before, during, and/or after insertion into bone. The device 10 can desirably improve bone purchase by being secured into more bone after implantation, for example, by being longitudinally lengthened along the device longitudinal axis 8_{L}. Such increases in bone purchase can improve the versatility of current attachment devices, lessen surgical time, produce less waste, and improve outcomes. By producing less waste, the device 10 can also advantageously decrease the number of stock keeping units within hospital systems. The increasable length of the device 10 can also desirably enable the device 10 to better resist migration from tensile loads and better resist left and right motion (windshield wiper effect), for example by increasing the resistance that the device 10 has to these types of motions when longitudinally expanded from a first length in a first configuration (e.g., unexpanded or partially expanded) to a second length greater than the first length in a second configuration (e.g., partially expanded or fully expanded). When in an expanded configuration, the device 10 can attach to more bone relative to when in an unexpanded or partially expanded configuration. When in an expanded configuration, the device 10 can attach to the same amount of bone relative to when in an unexpanded or partially expanded configuration, but can have a longer length. The lengthening device 10 can thereby better resist movement inside the bone when subject to long term dynamic motion, which can also decrease the probability of slippage or stripping. The increasable length of the device can also desirably allow the length of the device 10 to be adjusted according to each person's needs, for example, to fit the geometry of a medium such as a vertebra. Once the device 10 is implanted (e.g., implanted long term), the length of the device 10 can be decreased prior to removal (e.g., from bone), which can desirably decrease the forces needed to remove the device 10 from bone, for example, by allowing the decreasable length to be decreased first while the device 10 is still implanted, and then removing the device 10 once the device length is shortened, for example, from the second configuration back to the first configuration (or any two configurations where the first configuration is shorter than the second configuration).

FIG. 1A illustrates that the device 10 can have a device proximal end 10a and a device distal end 10b. The device distal end 10b can be moveable relative to the device proximal end 10a, or vice versa. For example, the device distal end 10b can be longitudinally moveable along the device axis 8_{L} relative to the device proximal end 10, or vice versa. The device proximal and distal ends 10a, 10b can be separately, sequentially, and/or simultaneously moveable relative to each other, for example, longitudinally along the device axis 8_{L}. For example, the device proximal end 10a can remain in a fixed position while the device distal end 10b is moved longitudinally away from the device proximal end 10a, or vice versa.

The device 10 can have one or more shafts 12, for example 1, 2, 3, 4, 5 or more shafts. The one or more shafts 12 can collectively form a frame of the device 10, with each individual shaft 12 forming part of the frame. For example, FIG. 1A illustrates that the device can have a first shaft 12a and a second shaft 12b (first and second shafts 12a, 12b are also referred to as proximal and distal shafts 12a, 12b, or with other relative positional identifiers where more than two shafts are used, for example, proximal, proximal-middle, middle, distal-middle, and distal shafts for devices having 5 shafts). The proximal shaft 12a (or a portion thereof) can define the device proximal end 10a and the distal shaft 12b (or a portion thereof) can define the device distal end 10b. The proximal shaft 12a can have a proximal shaft proximal terminal end 15a and a proximal shaft distal terminal end 15b. The distal shaft 12b can have a distal shaft proximal terminal end 16a and a distal shaft distal terminal end 16b.

The distal shaft 12b can be moveable relative to the proximal shaft 12a, or vice versa. For example, the distal shaft 12b can be longitudinally moveable along the device axis 8_{L} relative to the proximal shaft 12a, or vice versa. The proximal and distal shafts 12a, 12b can be separately, sequentially, and/or simultaneously moveable relative to each other, for example, longitudinally along the device axis 8_{L}. For example, the proximal shaft 12a can remain in a fixed position while the distal shaft 12b is moved longitudinally long the device axis 8_{L} such that the distal shaft distal terminal end 16b is moved longitudinally away from the proximal shaft distal terminal end 15b, or vice versa.

The device 10 can be deformable and/or non-deformable. The shafts 12 can be non-deformable. The shafts 12 can he deformable. The shafts 12 can be rigid, semi-rigid, flexible, or any combination thereof. The shafts 12 can be made from metal, plastic, or a composite material. For example, the shafts 12 can be made from titanium and/or steel. The proximal shafts 12a can be made from the same or a different material than the distal shaft 12b. The frame can have any combination of these features.

The device distal end 10b can have a tip 19. The tip 19 can be sharpened or otherwise configured to seat the device 10 in bone (e.g., with cutting teeth). The device proximal end 10a can have a proximal end cap 18. The cap 18 can have a substantially spherical configuration. A neck 17 can connect the cap 18 to the proximal shaft 12a. The cap 18, neck 17, and/or proximal shaft 12a can be monolithically formed, integrated with one another, and/or attached to one another. The cap 18, neck 17, and/or tip can be rigid, semi-rigid, deformable, non-deformable, flexible, or any combination thereof. The cap 18 can have a tool first attachment port 20a. An attachment tool (not shown) can be configured to engage with the tool first attachment port 20a to deploy the device 10.

The device 10 can have threads 12_{T} that can be screwed into bone. The threads 12_{T} can be between the device proximal and distal ends 10a, 10b (e.g., between the proximal shaft proximal terminal end 15a and the distal shaft distal terminal end 16b). The threads 12_{T} can be configured to screw the device 10 into bone in one or multiple configurations, for example, during one or more deployment stages. For example, the device 10 can have a deployment first stage (e.g., where the device 10 is inserted in bone in an unexpanded or partially expanded configuration, e.g., FIG. 1A), a deployment second stage (e.g., where the device 10 is longitudinally lengthened in bone, e.g., FIG. 1A to FIG. 1B), a deployment third stage (e.g., where the device 10 is longitudinally shortened in bone, e.g., FIG. 1B to FIG. 1A), a deployment fourth stage (e.g., where the device 10 is removed from bone, e.g., FIG. 1A), or any combination thereof.

One or more of the shafts 12 can have threads (e.g., threads 12_{T}). The shafts 12 can have outer threads 12_{OT} and/or inner threads 12_{IT}. The outer threads 12_{OT} can be configured to screw into bone. The inner threads 12_{IT} can be configured to axially expand and/or contract the device 10. Some threads 12_{T} can be internal threads 12_{IT} in a first configuration (e.g., unexpanded configuration) and external threads 12_{OT} in a second configuration (e.g., expanded configuration). A portion of the internal threads 12_{IT} can progressively become external threads 12_{OT} as the device 10 is longitudinally expanded, or vice versa. For example, the unexposed threads (e.g., internal threads 12_{IT}) can be configured to expand and/or contract the device 10 when engaged with another shaft 12 (e.g., with threads of another shaft), and can be configured to screw the device 10 into bone when not engaged with another shaft 12 (e.g., after having been progressively screwed out of the inside of the device 10). The internal threads 12_{IT} can progressively emerge from an inside of the device 10 when the device 10 is expanded.

FIG. 1A illustrates that the proximal and distal shafts 12 can have threads 12_{T} as shown when in an unexpanded configuration. For example, the proximal shaft 12a can have proximal shaft outer threads 12a_{OT} and the distal shaft 12b can have distal shaft outer threads 12b_{OT}. The proximal shaft outer threads 12a_{OT} and the distal shaft distal shaft outer threads 12b_{OT} can be configured to contact bone (e.g., when the device 10 is inserted and/or removed from bone). The proximal shaft outer threads 12a_{OT} and the distal shaft outer threads 12b_{OT} can be the same or different from one another. For example, the proximal shaft outer threads 12a_{OT} and the distal shaft outer threads 12b_{OT} can have the same dimensional quantities along the bodies of the proximal and distal shafts 12a, 12b and can be different, for example, along the taper 13 and near the tip 19.

The threads 12_{T} can have a thread pitch, for example, from about 0.25 mm to about 7.5 mm, including every 0.25 mm increment within this range, for example 1.0 mm. The threads 12_{T} can have a high thread pitch, for example, 5.0 mm. The threads 12_{T} can have a thread helix angle, for example, from about 1 degree to about 75 degrees, including every 1 degree increment within this range, for example 20 degrees. The threads 12_{T} can have a high thread pitch, for example, 5.0 mm. The threads 12_{T} can have a uniform or non-uniform thread. diameter along the length of the device 10. The threads 12_{T} can have a uniform or non-uniform thread depth along the length of the device 10. For example, FIG. 1A illustrates that the thread depth can decrease along a length of the device 10, for example where the device 10 has a taper 13. One or more portions of the device 10 can have a taper. For example, FIG. 1A illustrates that the proximal device end 10a can have the taper 13. The thread diameter along the length of the tapered section 13 can remain constant as shown in FIG. 1A, or can increase or decrease. The taper 13 can be configured to strengthen the attachment of the device 10 into bone by imparting a radial force onto the bone. This radial force can create compression that can be configured to prevent the device 10 from loosening once secured (e.g., screwed) into bone. This radial force can help create an interference fit (also referred to as a friction fit throughout) that can be configured to prevent the device 10 from loosening once secured (e.g., screwed) into bone. The taper 13 can have a taper angle 23 from about 1 degree to about 45 degrees, including every 1 degree increment within this range, for example, about 10 degrees.

FIG. 1A illustrates that the device 10 can have a longitudinally contracted configuration in which the device 10 can have a fully contracted length L_{C}. The fully contracted length L_{C} can be from about 15 mm to about 80 mm, including every 0.5 mm increment within this range, for example, about 53 mm. FIG. 1B illustrates that the device 10 can have a longitudinally expanded configuration in which the device 10 can have a fully expanded length L_{E}. The fully expanded length L_{E} can be from about 20 mm to about 100 mm, including every 0.5 mm increment within this range, for example, about 68 mm.

The attachment device 10 can be longitudinally expanded from a contracted configuration (e.g., from the fully contracted configuration of FIG. 1A) to an expanded configuration (e.g., to the fully expanded configuration of FIG. 1B) or to any partially expanded configuration therebetween (e.g., between full contraction and full expansion), for example as shown by arrow 100E in FIG. 1A. The attachment device 10 can be longitudinally contracted from an expanded configuration (e.g., from the fully expanded configuration of FIG. 1B) to a contracted configuration (e.g., to the fully contracted configuration of FIG. 1A) or to any partially contracted configuration therebetween (e.g., between full expansion and full contraction), for example as shown by arrow 100C in FIG. 1B. In this way the device 10 can be lengthened and/or shortened.

FIGS. 1A and 1B illustrate that one or more shafts 12 (or portions thereof) can fit into one or more other shafts 12 (or portions thereof). A portion of the distal shaft 12b can extend out of the proximal shaft 12a when the device 10 is expanded and a portion of the distal shaft 12b can extend into the proximal shaft 12a when the device 10 is contracted. When the device 10 is in an unexpanded configuration, a portion of the distal shaft 12b can be inside the proximal shaft 12a and/or outside of the proximal shaft 12a. When the device 10 is in an expanded configuration, more of the distal shaft 12b can be outside of the proximal shaft 12a than when the device 10 is in an unexpanded configuration.

FIGS. 1A and 1B further illustrate that the device 10 can have one or more telescopable shafts 12. The shafts 12 of the device 10 can partially and/or fully telescope relative to one another when the device 10 is expanded and/or contracted. The shafts 12 can telescope with one another such that the shafts 12 can be longitudinally translated along the device longitudinal axis 8_{L} by moving one or more first shafts (e.g., distal shaft 12b) into and/or out of another shaft (e.g., proximal shaft 12a), for example, via translational and/or rotational movement (e.g., a sliding or screwing motion). For example, the proximal and distal shafts 12a, 12b can telescope with each other such that the shafts 12a and/or 12b can be longitudinally translated along the device longitudinal axis 8_{L} by moving either or both of the shafts 12a and 12b into and/or out of another shaft, for example, via translational and/or rotational movement (e.g., a sliding or screwing motion).

The device 10 can be a telescoping attachment device 10, for example a telescoping screw 10 having one or more telescopable shafts 12. FIGS. 1A and 1B illustrate that the distal shaft 12b (or a portion thereof) can be configured to telescope into and out of the proximal shaft 12a. For example, the distal shaft 12b can have a distal shaft proximal portion 12b_{P} and a distal shaft distal portion 12b_{D}. The distal shaft proximal and distal portions 12b_{P}, 12b_{D} can be attached to or integrated with (e.g., monolithically formed) with each other. One or more portions of the distal shaft 12b can be telescopable into and/or out of the proximal shaft 12a, for example, such that the distal shaft 12b can fit in or be received by the proximal shaft 12a. For example, FIG. 1C illustrates that the distal shaft proximal portion 12b_{P} can fit into or be received by the proximal shaft 12a but not the distal shaft distal portion 12b_{D}. The distal shaft proximal portion 12b_{P} can be telescopable into and/or out of the proximal shaft 12a. The distal shaft distal portion 12b_{D} can remain external to the proximal shaft in both unexpanded and expanded configurations such that it is not telescopable within the proximal shaft 12a.

FIG. 1C illustrates that the proximal shaft 12a can have a proximal shaft proximal channel 28a and a proximal shaft distal channel 28b. The proximal shaft proximal channel 28a can be unoccupied (as shown) or can have or otherwise receive a portion of the distal shaft 12b. The proximal shaft proximal channel 28a can be configured to receive or otherwise engage with an attachment tool. The proximal shaft distal channel 28b can be configured to receive, house, or otherwise engage with the distal shaft proximal portion 12b_{P} when the device 10 is expanded, contracted, and/or when expansion and/or contraction is complete (e.g., when the device 10 is implanted and in a fully contracted configuration, a partially expanded configuration, or a fully expanded configuration). The proximal shaft distal channel 28b can be configured to progressively receive or otherwise engage with an attachment tool, for example, as the distal shaft 12b is longitudinally moved out of the device 10 when the device 10 is lengthened, and vice versa.

The distal shaft proximal portion 12b_{P} can be configured to telescope within the proximal shaft distal channel 28b but not the proximal channel proximal channel 28a. The distal shaft proximal portion 12b_{P} can be translated out of and/or into the proximal shaft distal channel 28b via translational and/or rotational movement (e.g., a sliding or screwing motion). For example, the distal shaft proximal portion 12b_{P} can have distal shaft inner threads 12b_{IT} and the proximal shaft 12a can have proximal shaft inner threads 12a_{IT}, for example in the proximal shaft distal channel 28b. The distal shaft inner threads 12b_{IT} and the proximal shaft inner threads 12a_{IT} can be configured to engage with one another. The distal shaft inner threads 12b_{IT} can be male threads and the proximal shaft inner threads 12a_{IT} can be female threads, or vice versa. The distal shaft inner threads 12b_{IT} can be for axial extension of the distal shaft 12b, where relative rotation between the proximal and distal shafts 12a, 12b via threaded engagement between the proximal shaft inner threads 12a_{IT} and the distal shaft inner threads 12b_{IT} can convert rotational motion into longitudinal expansion and/or contraction along the device longitudinal axis 8_{L}. The axial expansion threads 12a_{IT} can have the same or different pitch as the distal shaft bone threads 12b_{OT} and/or as the proximal shaft bone threads 12a_{OT}.

The proximal and/or distal shafts 12a, 12b can be rotated separately and/or simultaneously. As described above, the cap 18 can have a tool first attachment port 20a. FIG. 1C further illustrates that the proximal end of the distal shaft 12b can have a tool second attachment port 20b to which an attachment tool (not shown) can be configured to engage with. Alternatively or additionally, the tool first and second attachment ports 20a, 20b can be tool first and second attachment protrusions to which an attachment tool (not shown) can be configured to engage with. The proximal and distal shafts 12a, 12b can be configured to rotate simultaneously when an attachment tool (not shown) is attached to the tool first attachment port 20a, or to the tool first and second attachment ports 20a, 20b. The distal shaft 12b but not the proximal shaft 12a can be configured to rotate when the same or a different attachment tool (not shown) is attached to the tool second attachment port 20b. The proximal shaft 12a but not the distal shaft 12b can be configured to rotate when the same or a different attachment tool (not shown) is attached to the tool first attachment port 20a. The tool first and second attachment ports 20a, 20b can be concentric with one another or can be offset from one another. The tool first and second attachment ports 20a, 20b can be concentric with the device longitudinal axis 8_{L}.

The attachment tool can be configured to engage with the tool first attachment port 20a to deploy the device 10 into a first deployed position in which the device 10 is in an unexpanded configuration (e.g., when the device 10 is in the unexpanded configuration of FIGS. 1A and 1C). The attachment tool can simultaneously rotate the proximal and distal shafts 12a, 12b, for example, to screw the device 10 into bone into the first deployed position. The same or a different attachment tool can be configured to engage with the tool second attachment port 20b to deploy the device 10 into a second deployed position in which the device is in an expanded configuration (e.g., the fully expanded configuration of FIG. 1B or an expanded configuration in any 1 mm increment between the unexpanded configuration of FIG. 1A and the fully expanded configuration of FIG. 1B). When the attachment tool is engaged with the tool second attachment port 20b, the attachment tool can rotate the distal shaft 12b but not the proximal shaft 12a to lengthen the device 10 by rotating (e.g., screwing) the distal shaft 12b partially out of the proximal shaft distal channel 28b. The attachment tool can extend through proximal shaft proximal channel 28a to reach and engage with the tool second attachment port 20b. When the attachment tool is engaged with tool second attachment port 20b, the attachment tool can also be optionally engaged with the tool first attachment port 20a. The attachment tool can prevent rotation of the proximal shaft 12a and allow rotation of the distal shaft 12b when attached to both attachment ports 20a and 20b.

FIGS. 1A and 1C illustrate that the proximal shaft distal terminal end 15b can abut against the distal shaft proximal terminal end 16a at a seat 26 when the device 10 is in an unexpanded configuration. FIG. 1B illustrates that the proximal shaft distal terminal end 15b can be separated from the distal shaft proximal terminal end 16a by a gap when the device 10 is in an expanded configuration.

FIG. 1B further illustrates that the device 10 can have a device head length L_{H} (which includes the length of the cap 18 and the length of the neck 17 portion connecting the cap 18 to the proximal shaft 12a) of about 5 mm to about 15 mm, including every 0.5 mm increment within this range, for example, about 8 mm. The taper 13 can have a taper length L_{T} of about 5 mm to about 100 mm, including every 0.5 mm increment within this range, for example, about 10 mm. The proximal shaft 12a can have a proximal shaft length L_{P} of about 20 mm to about 100 mm, including every 0.5 mm increment within this range, for example, about 35 mm. The distal shaft 12b can have an expandable distal shaft length LED (also referred to as the exposable distal shaft length LED) of about 5 mm to about 80 mm, including every 0.5 mm increment within this range, for example, about 10 mm. The distal shaft proximal portion 12b_{P} can have an exposed distal shaft length L_{D1} when the device 10 is in a fully expanded configuration of about 5 mm to about 80 mm, including every 0.5 mm increment within this range, for example, about 5 mm. The exposed distal shaft length L_{D1} can correspond to the length by which the device 10 is expanded. The distal shaft distal portion 12b_{D} can have a distal shaft distal portion length L_{D2} (e.g., including the tip 19) of about 5 mm to about 80 mm, including every 0.5 mm increment within this range, for example, about 5 mm. FIG. 1C illustrates that the distal shaft 12b can have a distal shaft length L_{D} of about 20 mm to about 100 mm, including every 0.5 mm increment within this range, for example, about 20 mm. The distal shaft 12b can have a portion that remains in the proximal shaft 12a when the device 10 is fully expanded. As described in more detail below, a locking mechanism can prevent the distal shaft 12b from being expanded completely out of the proximal shaft 12a.

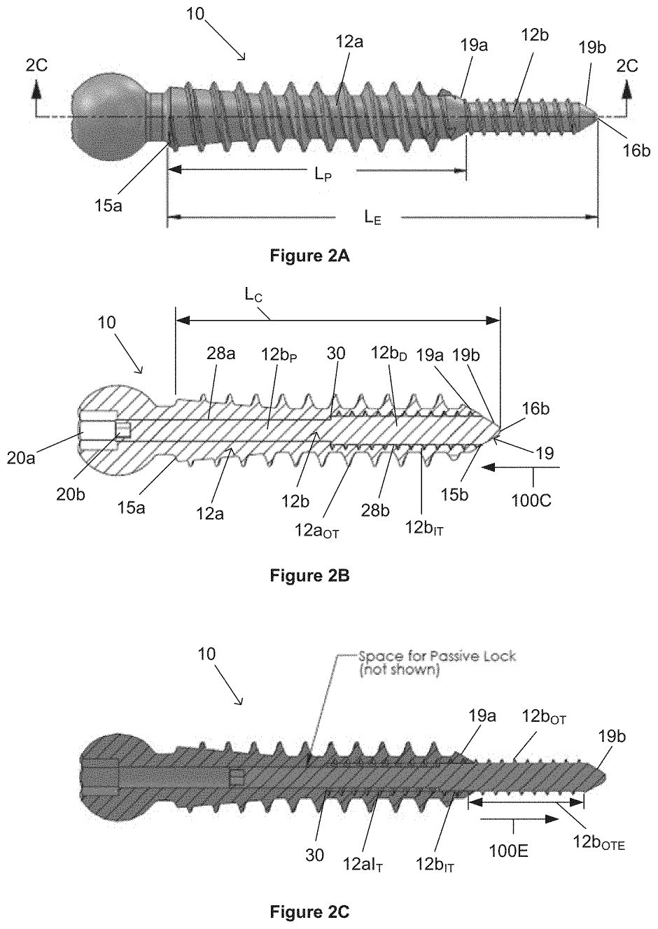

FIG. 2A illustrates that the fully expanded length L_{E} can be longitudinally measured from the proximal shaft proximal terminal end 15a to the distal shaft distal terminal end 16b. The fully expanded length L_{E} of the device of FIG. 2A can be from about 20 mm to about 100 mm, including every 0.5 mm increment within this range, for example, about 46.5 mm.

FIG. 2B illustrates that the fully contracted length L_{C} can be longitudinally measured from the proximal shaft proximal end 15a to the distal shaft distal terminal end 16b. The fully contracted length L_{C} of the device of FIG. 2B can be 15 mm to about 80 mm, including every 0.5 mm increment within this range, for example, about 37.0 mm.

FIGS. 2A-2C illustrates that the device 10 have a tip 19 having a proximal shaft tip portion 19a and a distal shaft tip portion 19b. The proximal and distal tip portions 19a, 19b can be configured to cut through bone when the device is screwed into bone in an unexpanded configuration. The distal tip portion 19b can be configured to cut through bone when the device 10 is lengthened into an expanded configuration, for example by translating the distal shaft 12b partially out of the proximal shaft 12a. The distal tip portion 19b but not the proximal tip portion 19a can be configured to cut through bone when the device 10 is lengthened into an expanded configuration.

FIGS. 2B and 2C illustrate that the distal shaft 12b can extend out of the proximal shaft 12a when the device 10 is expanded. The distal shaft 12b can fit into the proximal shaft 12a when the device 10 is contracted, for example, the entire distal shaft 12b (with the exception of the tip 19b) can fit within the proximal shaft 12a. The tip 19b can fit within the proximal shaft 12a, for example, such that the distal shaft distal terminal end 16b is flush or nearly flush with the proximal shaft distal terminal end 15b.

FIG. 2B illustrates that the distal shaft 12b can be in the proximal shaft proximal and distal channels 28a, 28b. The distal shaft proximal portion 12b_{P} can fit into or be received by the proximal shaft proximal channel 28a. The distal shaft distal portion 12b_{D} can fit into or be received by the proximal shaft distal channel 28b but not the proximal shaft proximal channel 28a. The distal shaft proximal portion 12b_{P} can be configured to telescope within the proximal shaft proximal and distal channels 28a, 28b. The distal shaft distal portion 12b_{D} can be configured to telescope within the proximal shaft distal channel 28b but not the proximal channel proximal channel 28a, with a size difference between the distal shaft distal portion 12b_{D} and the proximal shaft proximal channel 28a preventing the distal shaft distal portion 12b_{D} from longitudinally translating into the proximal shaft proximal channel 28a. For example, the distal shaft distal portion 12b_{D} can have a larger diameter than the proximal shaft proximal channel 28a. The proximal shaft 12a can have a flange 30 which prevents distal shaft 12b from being pushed into the proximal shaft 12a during insertion into bone.

FIG. 2B illustrates that the top of the tool second attachment port 20b can be flush or nearly flush with the bottom of the tool first attachment port 20a when the device 10 is in an unexpanded configuration.

FIGS. 2B and 2C illustrate that the device 10 can be longitudinally contracted and expanded as shown by contraction and expansion arrows 100C, 100E, respectively.

FIGS. 2B and 2C illustrate that the proximal shaft outer threads 12a_{OT} can be configured to screw the device 10 into bone when the device 10 is an unexpanded configuration. A portion of the distal shaft internal threads 12b_{IT} can progressively become exposed and become distal shaft external threads 12b_{OT} as the device 10 is longitudinally expanded, or vice versa. An exposed thread portion 12b_{OTE} is shown in FIG. 2C. The exposed thread portion 12b_{OTE} can correspond to the length by which the device 10 is expanded. The unexposed threads (e.g., the internal threads 12b_{IT} in FIG. 2B) can be configured to expand and/or contract the device 10 when engaged with the proximal shaft internal threads 12a_{IT}. The distal shaft external threads 12b_{OT} that are outside of the proximal shaft 12a can be configured to screw the device 10 into bone (e.g., after having been progressively screwed out of the inside of the proximal shaft 12a). The internal threads 12_{IT} can progressively emerge from an inside of the device 10 when the device 10 is expanded.

FIGS. 2A-2C illustrate that the proximal shaft outer threads 112a_{OT} and the distal shaft outer threads 12b_{OT} can be different from one another as shown. For example, the distal shaft outer threads 12a_{OT} can be finer/smaller than the proximal shaft outer threads 12b_{OT} so there is no reliance on thread pitch matching between the bone insertion threads 12a_{OT} and the distal expansion threads 12b_{OT}. The thread pitch, thread depth, outer thread dimeter, inner thread diameter, and/or thread helix angle of the bone insertion threads 12a_{OT} can be larger than the bone expansion threads 12b_{OT}.

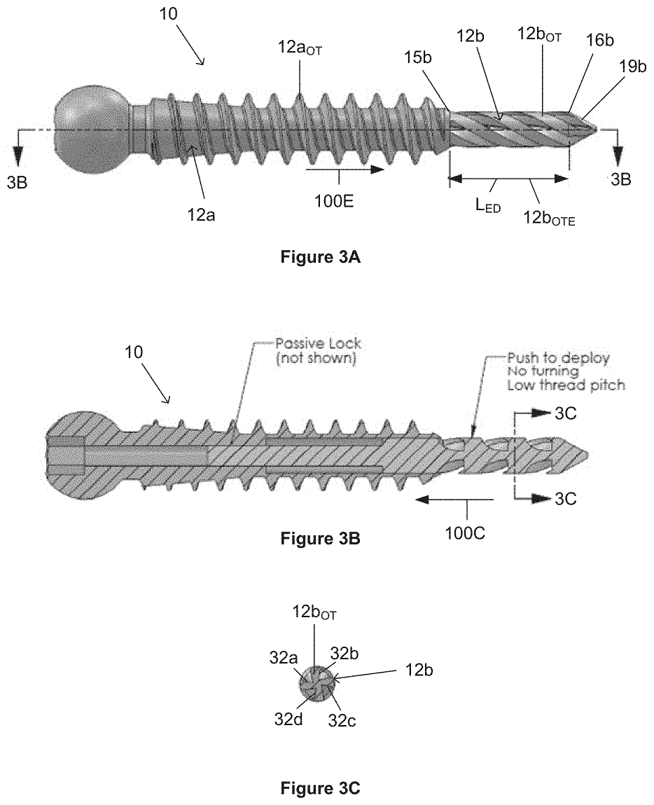

FIGS. 3A and 3B illustrate that the proximal shaft outer threads 12a_{OT} and the distal shaft outer threads 12b_{OT} can be different from one another. For example, the proximal shaft outer threads 12a_{OT} can have dimensional quantities similar to those described above with reference to the proximal shaft outer threads 12a_{OT} of FIGS. 1A-1C. The distal shaft outer threads 12b_{OT} can have a low thread pitch, for example, from about 2.0 mm to about 8 mm, including every 1.0 mm within this range, for example 4.0 mm. FIGS. 3A and 3B further illustrate that the distal shaft 12b can be pushable (as opposed to screwable). The low pitch of the distal shaft outer threads 12b_{OT} can allow the distal shaft 12b to rotate into and cut bone when an attachment device is used to push a proximal terminal end of the distal shaft 12b to expand the device 10 by longitudinally translating a portion of the distal shaft 12b out of the proximal shaft 12a.

The threads 12_{T} can have a thread helix angle, for example, from about 1 degree to about 75 degrees, including every 1 degree increment within this range, for example 45 degrees.

FIG. 3A illustrates that the expandable distal shaft length L_{ED} (also referred to as the exposable distal shaft length L_{ED}) L_{ED} can be longitudinally measured from the proximal shaft distal terminal end 15b to the distal shaft distal terminal end 16b. The expandable distal shaft length L_{ED} of the device 10 of FIG. 2A can be from about 5 mm to about 80 mm, including every 0.1 mm increment within this range, for example, about 12.9 mm.

The exposed thread portion 12b_{OTE} can have the same length as the expandable distal shaft length L_{ED} as shown in FIG. 1C.

FIG. 3C illustrates a variation of a transverse cross section of the distal shaft 12b showing that the distal shaft 12b can have four bone cutting edges 32a, 32b, 32c, and 32d.

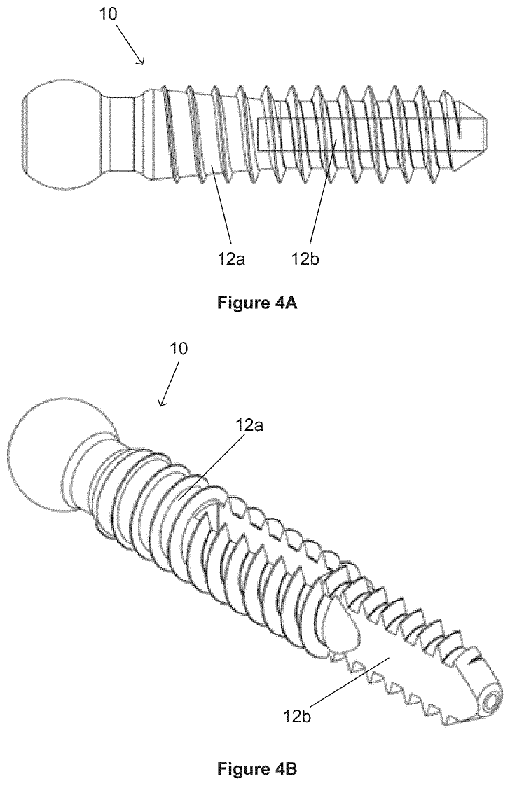

FIGS. 4A and 4B illustrate that the distal shaft 12b can slidably translate (e.g., without rotation) within the proximal shaft 12a. The distal section 12b can be pushable into bone. The distal threads 12b_{OT} can cut through bone, or displace bone, for example, cancellous bone (also referred to as spongy bone). Spongy bone can rebound into the gaps between the distal threads 12b_{OT} after insertion, thereby securing the device 10 into bone. Alternatively or additionally, the distal threads 12b_{OT} can be flexible in a first direction (e.g., expansion direction) and be inflexible or otherwise more rigid in a second direction opposite the first direction (e.g., contraction direction). In this way the bone can cause the distal threads 12b_{OT} to deflect toward the distal shaft 12b when the device is being longitudinally lengthened. The device 10 can be pulled in the second direction, e.g., slightly longitudinally contracted to cause the distal threads 12b_{OT} to “bite” into the bone.

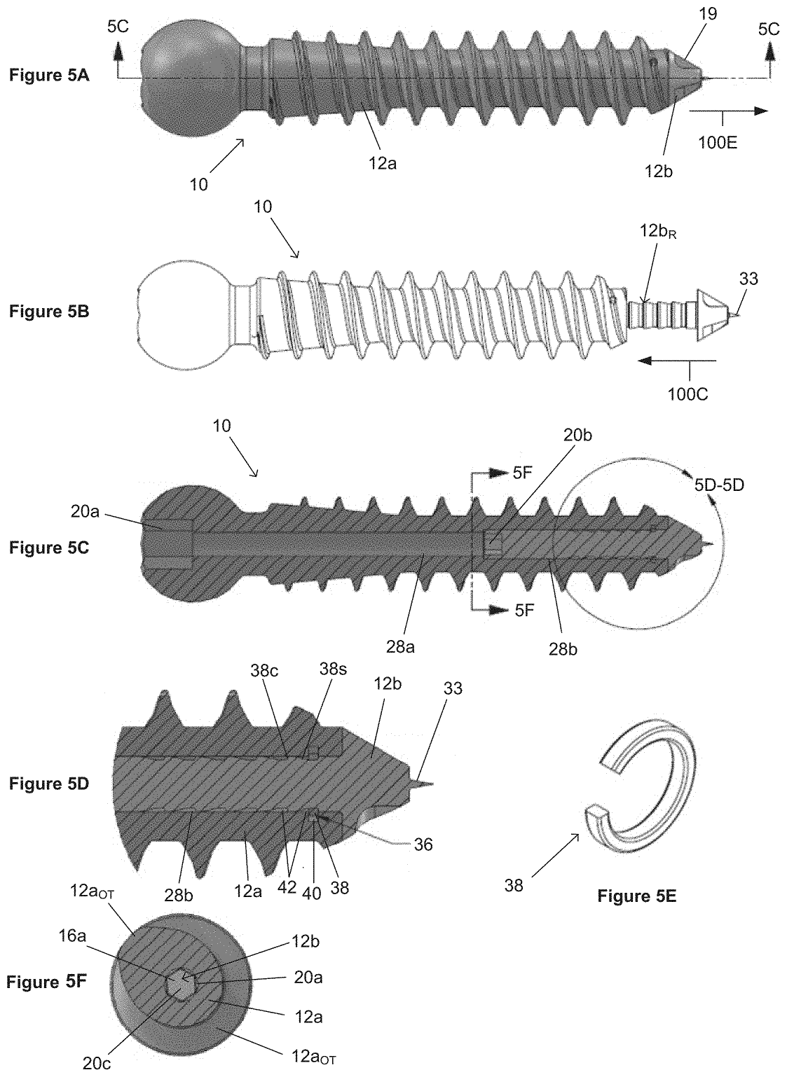

FIGS. 5A-5D illustrate that the distal shaft 12b can extend out of the proximal shaft 12a when the device 10 is expanded. The distal shaft 12b can fit into the proximal shaft 12a when the device 10 is contracted, for example, the entire distal shaft 12b (with the exception of the tip 19) can fit within the proximal shaft 12a. The tip 19 can fit within the proximal shaft 12a, for example, during removal of the device 10 from bone such that the distal shaft distal terminal end 16b is flush or nearly flush with the proximal shaft distal terminal end 15b. 33: anterior cortical purchase to increase toggle strength of screw. The tip 19 can have an anterior cortical purchase 33 (also referred to as a spike) to increase the toggle strength of the device 10. The spike 33 can help the tip 19 cut into bone. The device 10 can be a hi-cortical support with no threads on the distal shaft 12b.

FIGS. 5A-5D further illustrate that the distal shaft 12b can be pushable (as opposed to screwable) through the proximal shaft channel 28 (e.g., the proximal shaft distal channel 28b). The distal shaft can have one or more distal shaft ridges 12b_{R}, for example, 1 to 30 ridges, including every 1 ridge increment within this range, for example, 5 ridges. The distal shaft ridges 12b_{R} can be configured to engage with a locking mechanism 36 as the device 10 is expanded and the distal shaft 12b is longitudinally translated out of the proximal shaft 12a. The ridges 12b_{R} and the locking mechanism 36 can be a ratchet system such that the ridges 12b_{R} can be ratchetable with and/or through the locking mechanism 36. Alternatively or additionally, the locking mechanism 36 can comprise the ridges 12bR such that the locking mechanism 36 is a ratchet mechanism.

The locking mechanism 36 can include a stop 38 and a recess 40. The recess 40 can be a groove in the proximal shaft 12a. The stop 38 can be seated in the recess 40. The stop 38 can be a straight and/or curved length of material, for example, a ring or a semi-annular ring. The stop 38 can be elastic. For example, the stop 38 can be a spring (e.g., a pre-stressed material, or a shape-constrained material such as a ring 38 that has a smaller diameter than the recess 40 when in an unstressed or undeflected configuration). For example, the stop 38 can have a compressive strength of about 20 lb to about 150 lb, including every 1 lb increment within this range, for example, 121 lb. The recess 40 can be sized and shaped to receive the stop 38. For example, the recess 40 can be an annular or semi-annular recess to house and/or receive an annular or semi-annular stop 38. The stop 38 can have a deflected configuration and an undeflected configuration. FIG. 5D illustrates the stop 38 in an undeflected configuration in which the stop is only partially seated within the recess 40. The stop 38 can deflect further into the recess 40 (e.g., fully into the recess 40) when a ridge 12b_{R} passes over the stop 38 during expansion of the device. For example, the stop 38 can deflect radially outward from the device longitudinal axis 8_{L} when the ridge 12b_{R} passes over the stop 38. The stop 38 can snap back or otherwise return to its undeflected state from a deflected state after the ridge 12b_{R} passes over the stop 38. For example, the stop can deflect radially inward toward the device longitudinal axis 8_{L} after the ridge 12b_{R} passes over the stop 38. As the device is lengthened by pushing the distal shaft 12b longitudinally through the proximal shaft 12a, one or more ridges can pass over the stop 38. FIG. 5D illustrates that there can be one or more spaces 42 between the proximal shaft 12a and the distal shaft 12b for the stop 38 to be able to have an undeflected configuration. The spaces 42 can be between two or more adjacent ridges 12b_{R}. For example, the proximal and distal shafts 12a, 12b can cooperate to define 1 to 30 spaces 42, including every 1 space increment within this range, for example, 6 spaces 42 (with one space distal to the first ridge 12b_{R}).

The locking mechanism 36 can permit motion of the distal shaft 12b in a first direction and inhibit or prevent motion of the distal shaft 12b in a second direction opposite the first direction. For example, the locking mechanism 36 can permit the device 10 to expand and can inhibit or prevent the device 10 from contracting from an expanded configuration. The locking mechanism 36 can allow longitudinal expansion, for example, by allowing the ridges 12b_{R} of the distal shaft 12b to pass over the stop 38 in a first direction and by inhibiting or preventing the ridges 12b_{R} from passing over the stop 38 in the second direction. To achieve such motion, each ridge 12b_{R} can have a sloped surface 38s and a catch surface 38c. The sloped surface 38s can have a slope that can slide over and deflect the stop 38. The sloped surface 38s can have an angle from about 1 degree to about 70 degrees, including every 1 degree increment within this range, for example, 10 degrees (e.g., relative to the device longitudinal axis 8_{L}). The catch surface 38c can be flat or otherwise have a geometry configured to inhibit or prevent the ridge from passing over the stop 38 in the second direction (e.g., contraction direction). The catch surface 38c can have an angle from about 60 degrees to about 110 degrees, for example about 90 degrees (relative to the device longitudinal axis 8_{L}). Alternatively or additionally, the catch surface 38c can have a protrusion (not shown) and the distal side of the stop 38 can have a recess configured to receive the protrusion. The locking mechanism 36 can be a passive locking mechanism. The locking mechanism 36 can be unlocked with an actuating mechanism configured to deflect the stop 38 into the recess. The locking mechanism 36 can be configured to unlock by breaking. For example, upon removal of the device 10 from bone, a sufficient force can be applied which can cause the stop 38 to break apart or shear along a shear plane. The shear plane can be designed to not shear until the shear force reaches about 20 lb to about 150 lb, including every 1 lb increment within this range, for example, 50 lb. Alternatively or additionally, a stop dissolution substance can be communicated to the stop 38 through a stop dissolution channel. The stop 38 can dissolve and the distal shaft can be longitudinally compressed into the proximal shaft.

The device 10 can be removed with or without contracting (e.g., fully or partially) the device 10 before its removal from bone.

FIG. 5E illustrates a variation of the stop 38.

FIG. 5F illustrates that the distal shaft 12b can have a tool second attachment port and/or protrusion 20b having a curved and/or polygonal shape, for example, a hexagonal shape. FIG. 5F also illustrates that the proximal shaft outer threads 12a_{OT} can be integrated with or be monolithically formed with the proximal shaft 12a.

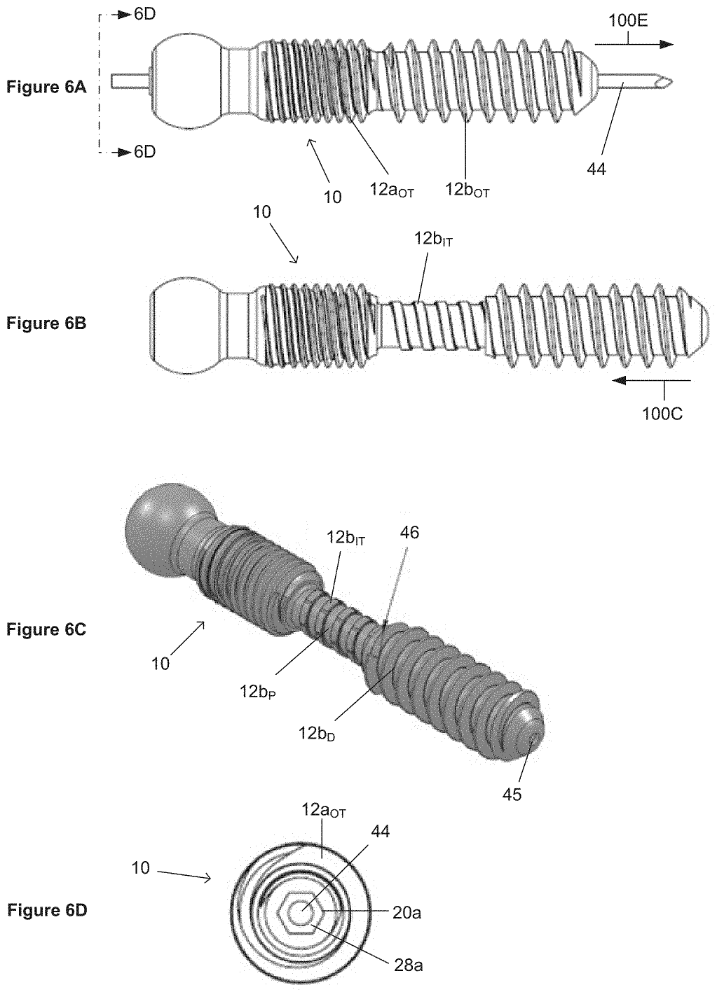

FIG. 6A illustrates that the device 10 can be cannulated. A bone spike 44 (also referred to as a needle) can extend through the device 10, for example, through the longitudinal center along the device longitudinal axis 8_{L}. This can be advantageous, for example, for MIS delivery over the needle 44. The bone spike 44 can be configured to stabilize the device 10 early on during the insertion process and/or throughout the insertion process. FIG. 6B illustrates that the bone spike 44 can be removed at some point during the insertion process, or after the device 10 has been fully implanted. FIGS. 6A and 6B illustrate that the proximal and distal shaft outer threads 12a_{OT}, 12b_{OT} can have different thread dimensions relative to one another. For example, FIGS. 6A and 6B illustrate that the proximal shaft outer threads 12a_{OT} can have a thread pitch that is less than that of the distal shaft outer threads 12b_{OT}. The proximal shaft outer threads 12a_{OT} can have a thread pitch, for example, from about 0.25 mm to about 7.5 mm, including every 0.25 mm increment within this range, for example 1.0 mm. The distal shaft outer threads 12b_{OT} can have a thread pitch, for example, from about 0.25 mm to about 7.5 mm, including every 0.25 mm increment within this range, for example 3.0 mm.

FIG. 6B illustrates that the distal shaft internal threads 12b_{IT} can have a flat thread surface (as opposed to a pointed surface when compared to, for example, the distal shaft internal threads 12b_{IT} illustrated in FIGS. 1B and 1C).

FIG. 6C illustrates that the threads 12_{T} can have one or more cutting flutes 46 (also referred to as screw removal cutting flutes) to aid in the removal of the device 10 from bone. The cutting flutes 46 can cut bone when the device 10 is removed from bone (e.g., rotated the opposite direction from the insertion rotation direction). The threads 12_{T} can have 1 to 20 cutting flutes 46, including every 1 cutting flute increment within this range, for example, 1 cutting flute. The proximal shaft outer threads 12a_{OT} and/or the distal shaft outer threads 12b_{OT} can have one or more cutting flutes 46. For example, FIG. 6C illustrates that the device 10 can have a cutting flute 46 on the proximal end of the distal shaft distal portion 12b_{D}.

FIG. 6C further illustrates a bone spike channel 45 with the bone spike 44 removed.

FIG. 6D illustrates that the proximal shaft 12a can have a tool first attachment port and/or protrusion 20a having a curved and/or polygonal shape, for example, a hexagonal shape. The bone spike 44 can pass through the center of the device 10. The bone spike 44 can be slidable within the proximal shaft proximal channel 28a and the proximal shaft distal channel 28b (not shown, as well as within one or more distal shaft channels (not shown, for example, a distal shaft proximal channel and/or a distal shaft distal channel).

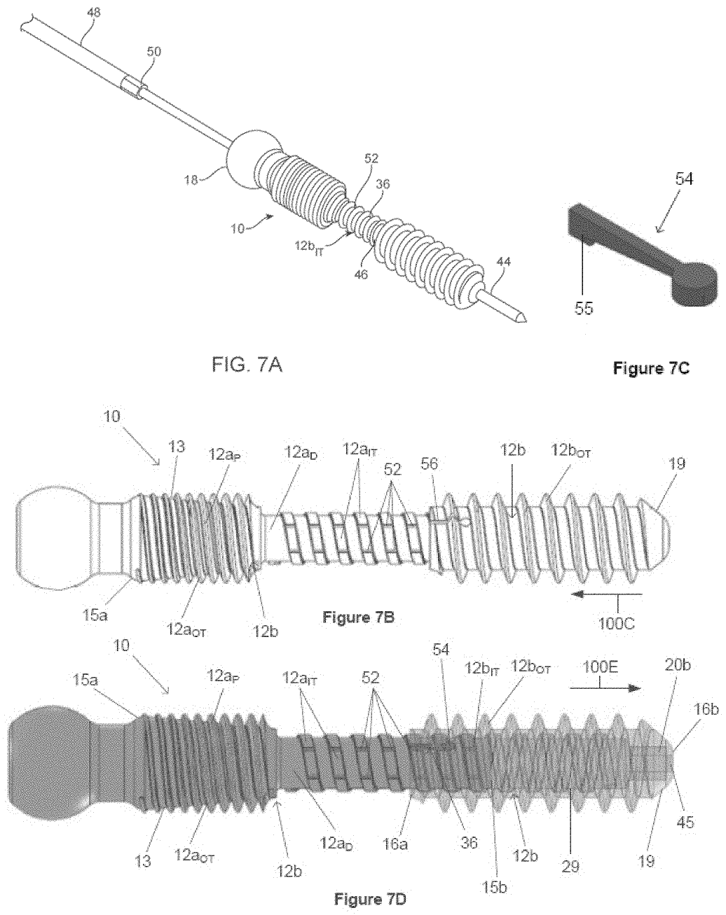

FIG. 7A illustrates that the system can have an attachment tool 48 removably attachable to the device 10. The attachment tool 48 can have a distal end having a radial driver 50 configured to axially expand the distal shaft 12b of the device 10. The attachment tool 48 can removably receive the bone needle 44. The radial drive 50 can be configured to engage with the tool first and/or second attachment ports 20a, 20b to axially translate the proximal shaft 12a and/or the distal shaft 12b in bone.

FIGS. 7A-7D illustrate that the locking mechanism 36 can include one or more locking grooves 52 and a stop 54. The locking grooves 52 can be one or more grooves on the proximal and/or distal shafts 12a, 12b, for example, on one or more of the outer and/or inner threads of each. For example, FIGS. 7A-7D illustrate that the distal shaft inner threads 12b_{IT} can have one or more locking grooves 52. The device 10 can have 1 to 50 locking grooves, including every 1 groove increment within this range, for example, 24 locking grooves as shown in FIG. 7B (12 are on the opposite side view and are not visible). The distal shaft inner threads 12b_{IT} can have a locking groove 52 along the length of the inner threads 12b_{IT} about every 1.0 mm to about 50.0 mm, including every 1.0 mm increment within this range, for example, about 10 mm. The locking grooves 52 can be spaced along the inner threads 12b_{IT} such that the device 10 can be locked about every 0.5 mm axial increment to about every 10 mm axial increment from the unexpanded configuration to the fully expanded configuration, including every about 0.5 axial increment within this range. Alternatively or additionally, the device 10 can have a single locking groove 52 configured to lock the device 10 in the fully expanded configuration.

FIGS. 7B and 7C illustrate a stop recess 56 and the stop 54, respectively. The stop recess 56 can have a shape that is complementary to the stop 54. The stop 54 can have a groove catch 55. The stop 54 can be elastic. For example, the stop 54 can be a spring. The stop 54 can be laser welded or press fit into the distal shaft 12b in the stop recess 56. For example, the stop 54 can have a compressive strength of about 20 lb to about 150 lb, including every 1 lb increment within this range, for example, 121 lb. The stop can have a groove catch 55. The groove catch 55 can deflect into the grooves 52 as the device 10 is axially expanded. The locking mechanism 36 can allow longitudinal expansion, for example, by allowing the locking grooves 52 of the distal shaft 12b to pass under the groove catch 55 in a first direction and by inhibiting or preventing the locking grooves 52 from passing under the groove catch 55 in a second direction opposite to the first direction. The groove catch 55 can deflect into the grooves 52 as the grooves 52 pass under the stop 54.

FIG. 7D illustrates the device 10 in a locked configuration with the stop 54 in the stop recess 56, and the groove catch 55 deflected into a locking groove 52.

FIGS. 7C and 7D further illustrate that the proximal shaft 12a (or a portion thereof) can be configured to telescope into and out of the distal shaft 12b. For example, the proximal shaft 12a can have a proximal shaft proximal portion 12a_{P} and a proximal shall distal portion 12a_{D}. The proximal shaft proximal and distal portions 12a_{P}, 12a_{D} can be attached to or integrated with (e.g., monolithically formed) with each other. One or more portions of the proximal shaft 12a can be telescopable into and/or out of the distal shaft 12b, for example, such that the proximal shaft 12a can fit in or be received by the distal shaft 12b. The distal shaft 12b can be telescopable over the proximal shaft distal portion 12a_{D} (e.g., translatable and/or rotatable). The device 10 can be lengthened for example, by axially moving telescoping) the distal shaft 12b over the proximal shaft distal portion 12a_{D} away from the proximal shall proximal terminal end 15a. The distal shaft 12b can be rotated and/or translated over the proximal shaft 12a (e.g., the proximal shaft distal portion 12a_{D}). For example, FIGS. 7C and 7D illustrate that the proximal shall distal portion 12a_{D} can fit into or be received by the distal shaft 12b but not the proximal shaft proximal portion 12a_{P}. The distal shaft proximal portion 12b_{P} can be telescopable into and/or out of the proximal shaft 12a. The proximal shall proximal portion 12a_{D} can remain external to the proximal shaft in both unexpanded and expanded configurations such that it is not telescopable within the proximal shaft 12a.

The proximal shaft 12a can have proximal shaft outer threads 12a_{OT} and the distal shaft 12b can have distal shaft outer threads 12b_{OT}. The proximal shaft outer threads 12a_{OT} and the distal shaft distal shaft outer threads 12b_{OT} can be configured to contact bone (e.g., when the device 10 is inserted and/or removed from bone). The proximal shaft outer threads 12a_{OT} and the distal shaft outer threads 12b_{OT} can be the same or different from one another. For example, the proximal shaft outer threads 12a_{OT} and the distal shaft outer threads 12b_{OT} can have the same dimensional quantities along the bodies of the proximal and distal shafts 12a, 12b and/or can be different, for example, along the taper 13 and near the tip 19.

The proximal shaft distal portion 12b_{D} can have proximal shaft inner threads 12a_{IT} and the distal shaft 12b can have distal shaft inner threads 12b_{IT}, for example in a distal shaft channel 28. The proximal shaft inner threads 12a_{IT} and the distal shaft inner threads 12b_{IT} can be configured to engage with one another. The proximal shaft inner threads 12a_{IT} can be male threads and the distal shaft inner threads 12b_{IT} can be female threads, or vice versa. The proximal shaft inner threads 12a_{IT} can be for axial extension of the distal shaft 12b, where relative rotation between the proximal and distal shafts 12a, 12b via threaded engagement between the proximal shaft inner threads 12a_{IT} and the distal shaft inner threads 12b_{IT} can convert rotational motion into longitudinal expansion and/or contraction along the device longitudinal axis 8_{L}. The axial expansion threads 12a_{IT} can have the same or a different pitch as the distal shaft bone threads 12b_{OT} and/or as the proximal shaft bone threads 12a_{OT}.

FIG. 7D further illustrates that the tool second attachment port and/or protrusion can be at the distal end of the distal shaft 12b, for example, near (e.g., within about 1 mm to about 15 mm, including every 1 mm increment within this range, for example, about within 7 mm) or at the distal shaft distal terminal end 16b. The tool second attachment port and/or protrusion 20b can be partially or completely within the tip 19. FIG. 7D illustrates that the bone spike channel 45 can pass through the center of the tool second attachment port and/or protrusion 20b. A portion of the bone spike channel 45 can extend distal to the tool second attachment port and/or protrusion 20b. FIG. 7D further illustrates that the distal shaft 12b can have a distal shaft channel 29. The distal channel 29 can be configured to receive the proximal shaft 12a (e.g., the proximal shaft distal portion 12a_{D}). The bone spike channel 45 and the distal shaft 29 can overlap within the distal shaft 12b.

FIGS. 7A, 7B and 7D illustrate that the device 10 can be locked in an expanded configuration (e.g., a fully expanded configuration). However, the device 10 can be locked in a partially expanded configuration as well.

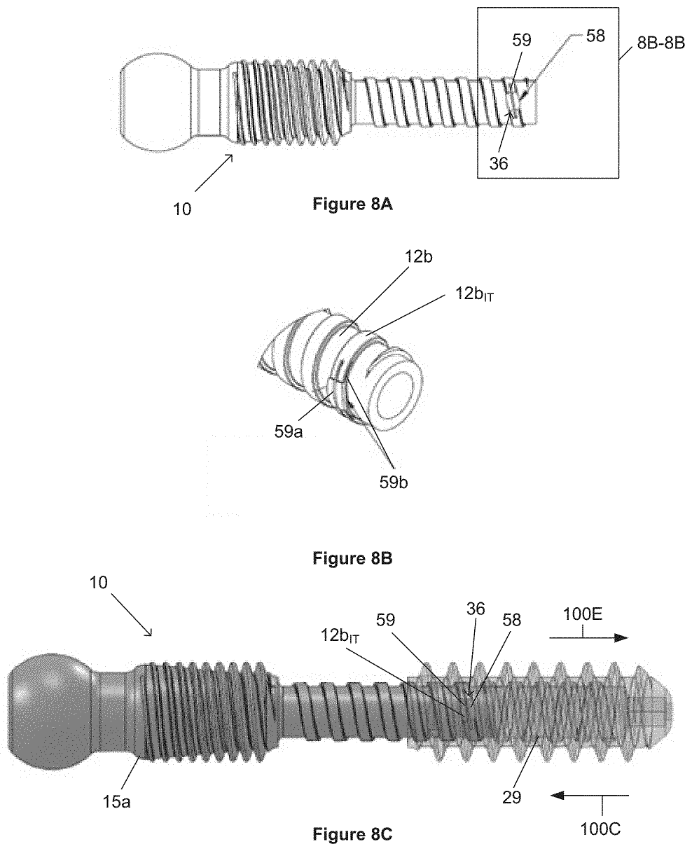

FIGS. 8A-8C illustrate that the locking mechanism 36 can include a thread 58 having one or more greater dimensions as compared to the surrounding threads on the proximal and/or distal shafts 12a, 12b, for example on one or more of the outer and/or inner threads of each. For example, FIGS. 8A-8C illustrate that the distal shaft inner threads 12b_{IT} can have one or more lock threads 58. The lock threads 58 can have a thread width that is greater than the surrounding inner threads 12b_{IT}, for example, by about 0.25 mm to about 2.0 mm, including every 0.25 mm increment within this range, for example, 1.0 mm. The lock threads 58 can have a thread height that is greater than the surrounding inner threads 12b_{IT}, for example, by about 0.25 mm to about 2.0 mm, including every 0.25 mm increment within this range, for example, 1.0 mm. The lock threads 58 can be lockable in a corresponding female thread via a friction fit or a snap fit. The corresponding female thread can be the distal shaft inner threads 12b_{IT} and can be configured to not allow the material to wide thread sections 58 to yield, thereby creating an outward force that can create a passive friction fit. This can desirably create a passive friction lock 36.

To create the lock fit, the distal shaft inner threads 12b_{IT} can have the width of the proximal shaft inner threads 12a_{IT} at each lock location. The remaining sections of the distal shaft inner threads 12b_{IT} can have the width and/or height of the lock threads 58. The lock threads 58 can be tapered such that each lock thread 58 has a wider base and a narrower apex.

The lock threads 58 can have one or more slots 59. For example the lock threads 58 can have a top slot 59a and/or a side or base slot 59b. The slots 59 (e.g., top slot 59a and/or side/bottom slot 59b) can extend along the length of the lock threads 58 and/or along a portion of the distal shaft inner threads 12b_{IT} on either or both sides of the lock threads 58. The slots 59 can have a slot length of about 1.0 mm to about 20 mm, including every 0.5 mm increment within this range, for example, about 4.0 mm. The slots 59 can have a slot length that is configured to not allow the material to yield, thereby creating an outward force for the creation of a passive friction lock, for example, any slot length having a dimension within the foregoing slot length range. The length of the top slot 59a can be the same or different from the bottom slot 59b (e.g., including greater than and/or less than). The side slots 59b can liberate the oversized lock threads 58.

The lock threads 58 can have a friction fit with one or more corresponding female thread sections of the distal shaft inner threads 12b_{IT} such that the lock threads 58 are wider and/or taller (e.g., have a greater depth or height) by about 0.25 mm to about 3.0 mm, including every 0.25 mm increment within this range, for example, about 0.5 mm.

The lock threads 58 (or lock thread portions) of the proximal shaft inner threads 12a_{IT} can be made of a less rigid material than the adjacent proximal shaft inner threads 12a_{IT}, which can allow the lock threads 58 to deform into a tighter friction fit. Such deformable lock threads 58 can be squeezed into the female lock thread portion. The corresponding female lock threads (not shown) of the distal shaft inner threads 12b_{IT} can likewise have a deformable or less rigid material, where the female threads can be configured to capture or otherwise lock with a lock thread 58 and/or with successive lock threads 58 as the distal shaft 12b is rotated relative to the proximal shaft 12a.

FIG. 8C further illustrates that the device can be in a partially expanded and/or contracted configuration such that the device 10 is expandable and/or contractible as shown by the expansion and/or compression arrows 100C, 100E. FIG. 8C illustrates that the device 10 can be locked in an expanded configuration (e.g., a fully expanded configuration). However, the device 10 can be locked in a partially expanded configuration as well.

The locking mechanism 36 can allow longitudinal expansion, for example, by inhibiting but allowing the lock threads 58 to rotate within and past the corresponding female threads (e.g., the distal shaft inner threads 12b_{IT} in a first direction and/or in a second direction opposite the first direction. The female threads can have a lockable portion configured to engage the lock threads 58 with a friction fit or a snap fit. The locking mechanism 36 can lock the proximal shafts 12a, 12b together when a lock thread 58 is left within a lockable portion of the female threads.

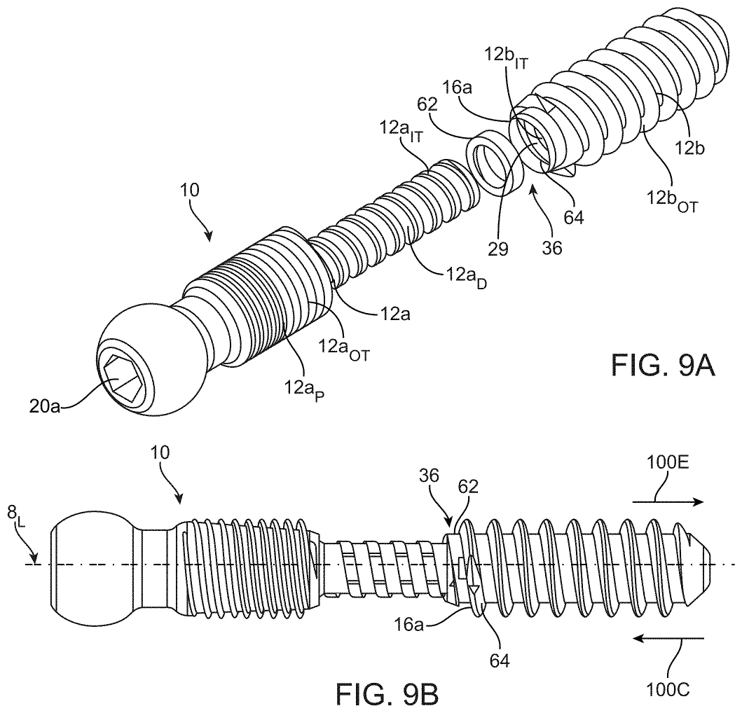

FIGS. 9A and 9B illustrate that the locking mechanism 36 can include a stop 62. The stop 62 can be in the distal shaft 12b. For example, the distal shaft 12b can have a stop recess 64 in which the stop 62 can reside. The stop 62 can be seated in the recess 64. The stop recess 64 can be at the distal shaft proximal end 16a. The stop 62 can be a straight and/or curved length of material, for example, a ring or a semi-annular ring. The recess 64 can be sized and shaped to receive the stop 62. For example, the recess 64 can be an annular or semi-annular recess to house and/or receive an annular or semi-annular stop 62. The stop 62 can be pinned against the inner wall of the distal shaft 12b. The stop 62 can be pinned against the top surfaces of the proximal shaft inner threads 12a_{IT}. The proximal shaft inner threads 12a_{IT} can be configured to interfere with the proximal shaft inner threads 12a_{IT}, which can generate drag (e.g., via an interference fit) to lock the device 10 together as the length of the device 10 is progressively increased by moving the distal shaft 12b axially along the device longitudinal axis 8_{L} away from the device proximal end (e.g., away from the proximal shaft proximal terminal end 15a). The locking mechanism 36 can allow longitudinal expansion, for example, by allowing but inhibiting the proximal shaft inner threads 12a_{IT} to pass under the stop 62 in a first direction and/or in a second direction opposite the first direction, such that the locking mechanism 36 can provide a friction fit for any position of the distal shaft 12b relative to the proximal shaft 12a during expansion and/or contraction of the device 10. FIG. 9B illustrates that the device 10 can be locked in an expanded configuration (e.g., a fully expanded configuration). However, the device 10 can be locked in a partially expanded configuration as well.

FIG. 9A further illustrates that the proximal shaft 12a can have a tool first attachment port and/or protrusion 20a having a curved and/or polygonal shape, for example, a hexagonal shape.

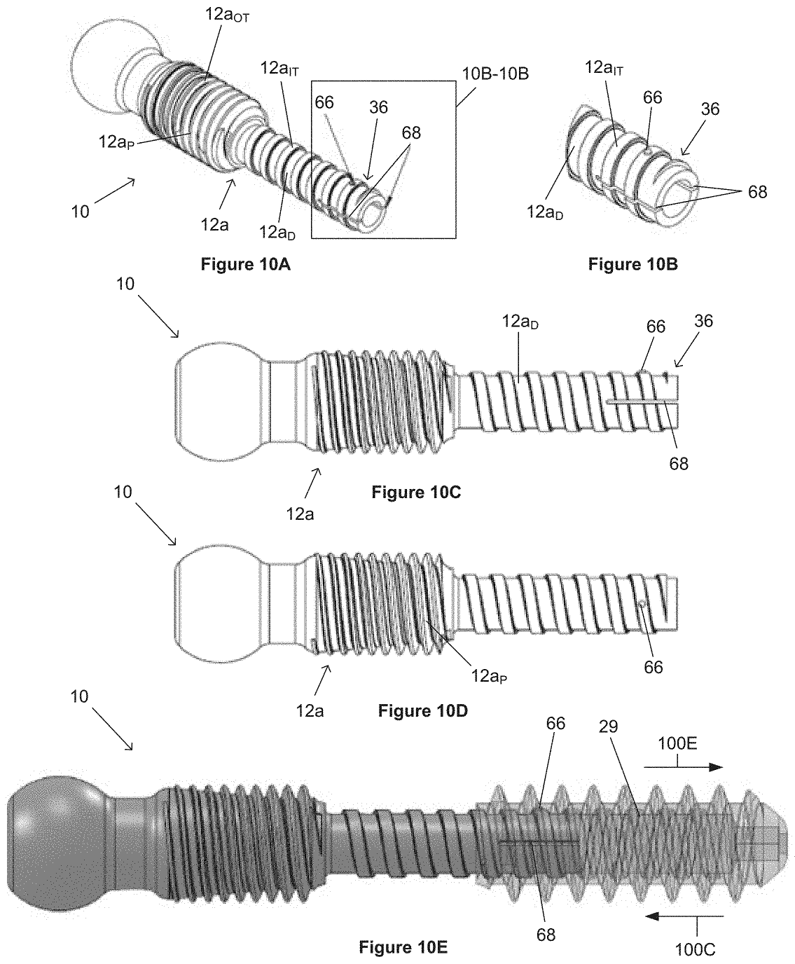

FIGS. 10A-10D illustrate that the locking mechanism 36 can include one or more friction bumpers 66. The device 10 can have 1 to 30 friction bumpers 66, including every 1 friction bumper increment within this range, for example, 1 friction bumper 66. The proximal shaft 12a can have one or more of the friction bumpers 66. For example, FIGS. 10A-10E illustrate that the friction bumpers 66 can be on the distal end of the proximal shaft 12a. The friction bumpers 66 can be made of a rigid or semi-rigid material. For example, the friction bumpers 66 can be made of titanium. The bumpers 66 can create a friction fit between the proximal and distal shafts 12a, 12b, for example by pressing against the distal shaft 12b. :For example, the bumpers can press against the distal shaft inner threads 12b_{IT} (male or female threads). The bumpers 66 can press against a side surface and/or against the base of the distal shaft inner threads 12b_{IT}. FIG. 110E illustrates that the bumpers 66 can be configured to press against the base of female distal shaft inner threads 12b_{IT}.

The locking mechanism 36 can have one or more shaft slots 68. The device 10 can have 1 to 30 shaft slots 68, including every 1 shaft slot increment within this range, for example, 2 shaft slots 68. The proximal shaft 12a can have one or more of the shaft slots 68. For example, FIGS. 10A-10D illustrate that the shaft slots 68 can be on the distal end of the proximal shaft 12a. The two shaft slots 68 can be on opposite sides of the device 10 such that they are separated by 180 degrees. The shaft slots 68 can be separated by any angle between about 10 degrees and about 180 degrees, including every 1 degree increment within this range, for example, about 180 degrees. The shaft slots 68 can be parallel to the device longitudinal axis 8_{L}. The shaft slots 68 can be non-parallel to the device longitudinal axis 8_{L}. For example, one or more shaft slots 68 can helically extend at least partially around a shaft 12 having one or more shaft slots 68. For example, the shaft slots 68 can helically extend around the proximal shaft distal portion 12a_{P}. The shaft slots 68 can configured to desirably allow an outside load (e.g., a compressive force) to be transferred from the bumper(s) 66 to inside of the distal screw section, e.g., to the inside of distal shaft 12b. The locking mechanism 36 can allow longitudinal expansion, for example, by allowing but inhibiting the bumper 66 to pass within female threads (e.g., the distal shaft inner threads 12b_{IT}) in a first direction and/or in a second direction opposite the first direction, such that the locking mechanism 36 can provide a friction fit for any position of the distal shaft 12b relative to the proximal shaft 12a during expansion and/or contraction of the device 10.

FIG. 10E illustrates that the device 10 can be locked in an expanded configuration (e.g., a fully expanded configuration). However, the device 10 can be locked in a partially expanded configuration as well.

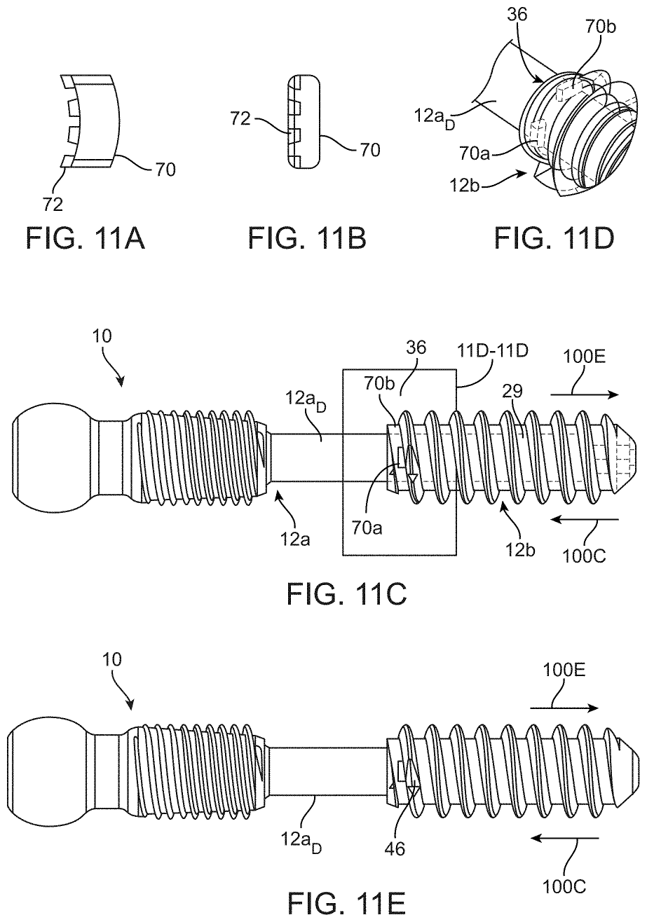

FIGS. 11A-11E illustrate that the locking mechanism 36 can include one or more inserts 70. The device 10 can have 1 to 10 inserts 70, including every 1 insert increment within this range, for example, 1 insert 70. The inserts 70 can have one or more protrusions 72 (also referred to as fingers). The inserts 70 can each have 1 to 10 protrusions 72, including every 1 protrusion increment within this range. For example, FIGS. 11A and 11B illustrate that the inserts 70 can each have four protrusions 72. When the device 10 has multiple inserts 70, different inserts can have the same or a different number of protrusions 72 as one another. FIG. 11A illustrates that the inserts 70 can have a curve to accommodate the cylindrical shafts 12. The inserts 70 can have a semi-annular shape. The inserts 70 can have an annular shape. The protrusions 72 can be configured to flex when the device 10 is longitudinally expanded to allow the shafts 12 to move relative to one another. The protrusions 72 can be configured to dig into a surface of the proximal and/or distal shafts 12a, 12b when the device 10 is in a locked configuration. The inserts 70 can be configured to produce a friction fit between the proximal and distal shafts 12a, 12b. When the protrusions 72 dig into a surface of the proximal and/or distal shafts 12a, 12b, the protrusions 72 can form load bearing columns when tensile loads are applied to the device 10. The protrusions 72 can have a hardness and clearance within the device to allow for such flexing and digging in of the protrusions 72. For example, the protrusions 72 can be a rigid or semi-rigid material. The inserts 70 can also be oriented 90 degrees to act as a one-way radial lock, for example, 90 degrees

The inserts 70 can be attached to or integrated with the device 10. For example, FIG. 11C illustrates that the inserts 70 can be attached to or integrated with the distal shaft 12b, for example, an inner surface of a recess or channel of the distal shaft 12b. The inserts 70 can be welded to the device 10, for example, to the distal shaft 12b. The inserts 70 can be positioned in one or more transversely and/or longitudinally extending planes. The inserts 70 can be space radially and/or longitudinally apart from one another. Radially adjacent inserts 70 can be positioned about 10 degrees to about 180 degrees apart from one another, including every 1 degree increment within this range, for example, 90 degrees. Longitudinally adjacent inserts 70 can be positioned about 1 mm to about 50 mm apart from one another, including every 1 mm increment within this range, for example, 5 mm.

FIG. 11C illustrates that the device 10 can have four inserts 70, with first and second inserts 70a and 70b visible. The inserts 70a, 70b, 70c (not visible), 70d (not visible) can be spaced apart by 90 degrees in as shown, with inserts 70a and 70b being adjacent to one another. The inserts 70a-70d can be in the same transverse plane. The third insert 70c can be opposite the first insert 70a and the fourth insert 70d can be opposite the second insert 70b. The device 10 of FIGS. 11A-11E can also only have the first and second inserts 70a, 70b, without the third and fourth inserts 70c, 70d.

FIG. 11C illustrates that that the protrusions 72 can be configured to engage with the proximal shaft 12a, for example, with the proximal shaft distal portion 12a_{D}. The proximal shaft distal portion 12a_{D} can be smooth. The proximal shaft distal portion 12a_{D} can have no threads (e.g., no proximal shaft inner threads 12a_{IT}).

FIGS. 11A-11E illustrate that the distal shaft 12b can be deployed by turning and/or pushing the distal shaft 12a over the proximal shaft proximal portion 12a_{D} with an attachment tool (not shown).

FIG. 11E further illustrates that the inserts 70 can be inside the device 10, for example, between an inner surface of the distal shaft 12b and the surface of the proximal shaft proximal portion 12a_{D}.

### Method of Use

The device 10 can have one or multiple configurations, one or more of which can correspond to various deployment stages or the transition from one stage to another. As described above, the device 10 can have a deployment first stage (e.g., where the device 10 is inserted in bone in an unexpanded or partially expanded configuration, e.g., FIG. 1A), a deployment second stage (e.g., where the device 10 is longitudinally lengthened in bone, e.g., FIG. 1A to FIG. 1B), a deployment third stage (e.g., where the device 10 is longitudinally shortened in bone, e.g., FIG. 1B to FIG. 1A), a deployment fourth stage (e.g., where the device 10 is removed from bone, e.g., FIG. 1A), or any combination thereof.

Additional deployment stages can include repositioning the device 10 (e.g., lengthening and/or shortening) in bone after an initial placement but prior to removal or a removal procedure. Additional deployment stages can include allowing bone to grow into the device 10 through one or more holes. Additional deployment stages involving longitudinal adjustment of the device 10 can be included where the device 10 has more than two shafts 12.

In the deployment first stage, the threads 12_{T} along the length of the device 10 can be progressively screwed into bone (e.g., progressively from the device distal end 10b to the device proximal end 10a). The deployment first stage can be completed when a majority of the threads 12_{T} (e.g., all of the threads 12_{T}) are screwed into bone, or such that the proximal shaft proximal terminal end 15a is seated within the bore created by the device 10, is seated flush with at least a portion of an osteo surface (e.g., vertebra surface), or is seated above an osteo surface (e.g., by about 0.5 mm to about 2.0 mm). The device 10 can be in an unexpanded configuration or in a partially expanded configuration throughout the deployment first stage, or only during a portion thereof. It can be advantageous to partially expand the device 10 during the deployment first stage to enable the device 10 to bite into or otherwise purchase more bone, which can be useful where the bone is brittle to provide additional stability during insertion. In the deployment first stage, the device distal and proximal ends 10b, 10a (e.g., the device distal and proximal shafts 12b, 12a) can both be longitudinally moveable in the bone by virtue of being screwed into the bone. Rotation of the device 10 (e.g., rotation of the distal and proximal shafts 12b, 12a) can cause the device distal and proximal ends 10b, 10a to simultaneously translate along the device longitudinal axis 8_{L} (with the threads converting rotational motion into translational motion). A first thread length can be screwed into bone during the deployment first stage, which can extend along the device distal and proximal ends 10b, 10a, for example, along a portion of the length (e.g., the entire length) of the distal and proximal shafts 12b, 12a, or along a portion of the length (e.g., the entire length) of the exposed/outward facing portions of the distal and proximal shafts 12b, 12a, where the exposed portions can be surfaces configured to contact bone during insertion (e.g., all of the shaft 12 surfaces and threads 12_{T} illustrated in FIG. 1A). The first thread length can progressively increase from an initial length (e.g., zero) to a final length (e.g., the length of the distal and proximal shafts 12b, 12a shown in FIG. 1A). The first thread length can correspond to the device fully contracted length L_{C}.

In the deployment second stage, the device 10 can be lengthened, for example from the device fully contracted length L_{C} to the device fully expanded length L_{E}. For example, the device distal end 10b (e.g., the distal shaft 12b or a portion thereof) can be rotated to lengthen the device 10. During rotation of the distal shaft 12b, the proximal shaft 12a can remain fixed, thereby allowing the device 10 to longitudinally expand along the device longitudinal axis 8_{L}. In the deployment second stage, a second thread length less than the first thread length can be screwed into bone, such that less of the device 10 is rotated in the deployment second stage than in the deployment first stage (e.g., the distal and proximal shafts 12b, 12a can be rotatable in the deployment first stage, whereas only the distal shaft 12b can be rotatable in the deployment second stage). The device 10 can be in a partially expanded configuration or in a fully expanded configuration at the end of the deployment second stage, with the final length of the device 10 being dependent on the dimensions of the bone into which the device 10 is screwed. In the deployment second stage, only the device distal end 10b (e.g., only the device distal shafts 12b) can be longitudinally moveable in the bone by virtue of being screwed into the bone. Rotation of the distal shaft 12b can cause the device distal end 10b to translate along the device longitudinal axis 8_{L} (with the threads converting rotational motion into translational motion) and lengthen the device 10 from a first length (e.g., length L_{C}) to a second length (e.g., length L^{E}) longer than the first length. The second thread length can remain constant as shown in FIGS. 1A and 1B, or can progressively increase as shown in FIGS. 2B and 2C.

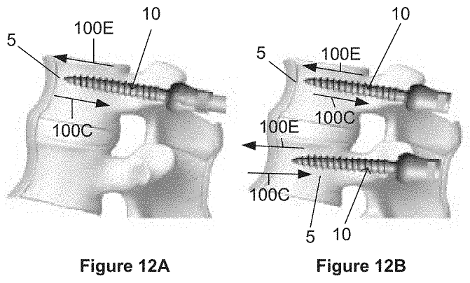

FIGS. 12A and 12B illustrate the attachment devices 10 of FIGS. 1A-11E implanted in various vertebrae 5 in expanded configurations. The devices 10 can be implanted in the vertebral bodies shown in FIGS. 12A and 12B. For example, the device 10 can be a longitudinally expandable and/or contractible screw such as a polyaxial pedicle screw that can be used as part of a spinal fixation system (e.g., to attach a rod and/or a plate of a fixation system to a vertebra).

In the deployment third stage, the device 10 can be shortened, for example, from the fully expanded length L_{E}, to the fully contracted length L_{C}, for example, by reversing the steps of the deployment second stage.

In the deployment fourth stage, the device 10 can be removed from bone, for example, by reversing the steps of the deployment first stage.

All references including patent applications and publications cited herein are incorporated herein by reference in their entirety and for all purposes to the same extent as if each individual publication or patent or patent application was specifically and individually indicated to be incorporated by reference in its entirety for all purposes. Many modifications and variations of this invention can be made without departing from its spirit and scope, as will be apparent to those skilled in the a

The specific embodiments described herein are offered by way of example only. Moreover, such devices and methods may be applied to other sites within the body. Modification of the above-described assemblies and methods for carrying out the invention, combinations between different variations as practicable, and variations of aspects of the invention that are obvious to those of skill in the art are intended to be within the scope of the claims.

1. An attachment device having a device longitudinal axis, comprising:
a longitudinally expandable length comprising a non-deformable frame having a first shaft and a second shaft, wherein the first shaft is telescopable within the second shaft.

2. The device of claim 1, wherein at least a portion of the first shaft is distal to at least a portion of the second shaft.

3. The device of claim 1, wherein the longitudinally expandable length is configured to longitudinally expand when the first shaft is translated in a first direction long the device longitudinal axis.

4. The device of claim 1, wherein the first shaft is engageable with the second shaft such that less of the first and second shafts are engaged with one another in a second configuration than in a first configuration.

5. The device of claim 4, wherein the first configuration comprises an unexpanded configuration of the longitudinally expandable length, and wherein the second configuration comprises an expanded configuration of the longitudinally expandable length.

6. The device of claim 5, wherein the expanded configuration comprises a fully expanded configuration of the longitudinally expandable length.

7. The device of claim 5, wherein the longitudinal expandable length is longitudinally contractible when the longitudinal expandable length is in the expanded configuration.

8. The device of claim 7, wherein the longitudinally expandable length is configured to longitudinally contract when the first shaft is translated in a second direction opposite the first direction along the device longitudinal axis.

9. The device of claim 1, wherein the frame comprises screw threads.

10. The device of claim 9, wherein the first shaft comprises first shaft bone threads and first shaft telescoping threads, and wherein the second shaft comprises second shaft bone threads and second shaft telescoping threads.

11. The device of claim 10, wherein the first and second shaft bone threads are male threads configured to screw into bone, wherein the first shaft telescoping threads are male or female threads, wherein the second shaft telescoping threads are the other of male or female threads, and wherein the first and second shaft telescoping threads are engageable with one another during longitudinal expansion of the device.

12. The device of claim 11, wherein the first and second shaft telescoping threads are engageable with one another during longitudinal expansion of the device.

13. The device of claim 11, wherein the device is a longitudinally expandable pedicle screw.

14. A method for deploying an attachment device having a device longitudinal axis, the method comprising:
inserting the attachment device of claim 1 into bone; and
longitudinally expanding a longitudinally expandable length of the attachment device.

15. The method of claim 14, wherein inserting comprises inserting into a vertebral body.

16. The method of claim 14, wherein longitudinally expanding comprises rotating the first shaft relative to the second shaft.

17. The method of claim 16, wherein longitudinally expanding comprises translating the first shaft relative to the second shaft.

18. The method of claim 14, wherein longitudinally expanding comprises rotating at least a portion of the frame about the device longitudinal axis in a first rotation direction.

19. The method of claim 18, wherein longitudinally expanding comprises translating at least a portion of the frame in a first translation direction along the device longitudinal axis.

20. The method of claim 19, further comprising longitudinally contracting the longitudinal length of the attachment device, wherein longitudinally contracting comprises at least one of rotating at least a portion of the frame about the device longitudinal axis in a second rotation direction opposite the first rotation direction and translating at least a portion of the frame in a second direction opposite the first direction along the device longitudinal axis.
